(19) United States
(12) Patent Application Publication
(10) Pub. No.: US 2008/0139512 A1
(43) Pub. Date: Jun. 12, 2008

(54) Method for minimizing or avoiding adverse effects of vesicants

(75) Inventors: David S. Lerner, Gainesville, FL (US); Gregory Schultz, Gainesville, FL (US)

Correspondence Address: ELMAN TECHNOLOGY LAW, P.C., P. O. BOX 209, SWARTHMORE, PA, 19081, US

(73) Assignees: Quick-Med Technologies, Inc., Gainesville, FL (US); University of Florida Research Foundation, Inc., Gainesville, FL (US)

(21) Appl. No.: 11/343,024

(22) Filed: Jan. 30, 2006

Related U.S. Application Data
(63) Continuation of application No. 10/256,215, filed on Sep. 25, 2002, now abandoned.

(51) Int. Cl. A61K 31/665; A61K 31/573; A61K 31/405; A61K 31/137; A61K 31/095

(52) U.S. Cl. 514/100; 514/171; 514/420; 514/649; 514/706

(57) ABSTRACT

The invention pertains to compositions and methods to treat the adverse effects of mustard chemicals and other toxic compounds, such as chemical warfare agents, exposure to which normally induces vesicating type response in mammals. In a rodent eye model at fixed concentrations of such a vesicant, compositions comprising a matrix metalloproteinase inhibitor, MMPI, a significant reduction in morbidity is achieved. with increased concentrations of the compositions of this invention, as compared with vehicle alone. Furthermore, compositions comprising the MMPI and in addition, an anti-inflammatory compound, in a vehicle appropriate to the type of tissue damage to be protected against from vesicant exposure, achieves both reduction in total tissue damage and inflammation, as compared with anti-inflammatory composition alone. Chemicals having more than one property, such as MMPI and AIA properties, are also disclosed.

### CROSS REFERENCE TO RELATED APPLICATIONS

This application is a continuation of and claims the benefit of priority from U.S. patent application Ser. No. 10/256,215, filed Sep. 25, 2002.

### FIELD OF THE INVENTION

Disclosed are compositions and methods for minimizing or avoiding adverse effects of vesicants such as mustard gas and other agents that cause vesication-type reactions. The compositions comprise at least one matrix metalloproteinase inhibitor and optionally include other disclosed biologically active compounds, such as an anti-inflammatory agent, biologically inert compounds, and combinations thereof.

### BACKGROUND OF THE INVENTION

The use of sulfur mustard, bis-(2-chloroethyl) sulfide, chloroethylethyl sulfide (CEES), and other vesicating agents in chemical warfare has been long known. More recently, use of such biohazards in the Iran-Iraq conflict resulted in many deaths and untold suffering. Use of such material was a major threat in the Gulf War. Hence, a composition and method for minimizing or preventing injury due to mustards and similarly acting chemical toxicants, particularly chemical warfare agents, is an important pursuit for scientists working for the U.S. Department of Defense. To date, identification of an appropriate and effective antidote and/or treatments have been pursued without noted success.

It is believed that nitrogen and sulfur mustard-induced vesication are due to the disruption of the connective tissue. In particular, it is believed that these and similar agents, including other chemical warfare agents, produce adverse effects through the separation of the epidermis from the dermis, and/or related types of tissue damage, as in cutaneous degradation and blister formation, damage to the cornea of the eye, mucous membranes, tracheal lining, bronchia, alveoli, and other internal and external tissues. As used throughout this disclosure, the terms vesicant, vesication-causing agent or chemical, vesicating agent, and the like, are taken to mean mustard chemicals as specifically enumerated herein, and other compounds, such as toxins and/or chemical warfare agents, that produce blistering upon contact with the skin of a living mammal.

U.S. Pat. No. 6,124,108 (the '108 patent) discloses a method for identifying induction or activation of a specific protease upon exposure to mustards. However, that patent acknowledges that no appropriate and effective antidote has been identified to date. Thus, a need exists for effective treatments and/or prophylactic preparations to deal with accidental or combat exposure to vesicating chemicals.

The '108 patent discloses that immunohistochemical studies have been conducted on the protein composition changes in mustard-exposed hairless guinea pig skin. Epidermal-dermal junction proteins, namely, bullous pemphigoid antigen, laminin and hemidesmosomal anchoring filament proteins were affected by exposure to mustard. More recently, investigators have found that in the mini pig skin, which is more akin to human skin, only one protein in the lamina lucida, the laminin, is affected by mustard exposure. These findings suggest that one or more specific protease(s) may be involved with the vesication induced by mustard and related vesicating chemicals.

The concept that a specific protease is involved in pathology related to exposure to mustards is important because the use of generalized protease inhibitors could cause serious side effects. Cowman, et al. have demonstrated that mustard gas and chloroethyl ethyl sulfide (CEES) stimulate protease activity in vitro in human peripheral blood lymphocytes and in vivo in hairless guinea pig skin. However, a definitive characterization of the mustard-stimulated protease is said to be described for the first time in the '108 patent.

The '108 patent discloses a separation and partial characterization of a protease which can be stimulated by exposure of NHEK cells to mustard in the presence of calcium ions. The protease so stimulated was completely inhibited by adding calcium chelator EGTA (2 mM), or serine protease inhibitor DFP (1 mM), or protein synthesis inhibitor cycloheximide (35.mu.M) in the extracellular medium. Proteolytic activity was also reported to be inhibited by leupeptin at 1 mM concentration but is not inhibited by pepstatin at 1 mM concentration. While the concentrations of these protease inhibitors substantially exceed the range of typical IC₅₀ values for such inhibitors, these results were interpreted to identify a serine protease that was stimulated by the mustard chemical.

The purportedly relevant protease was characterized in the '108 patent by electrophoretic separation (sodium dodecyl sulfate polyacrylamide gel electrophoresis (SDS-PAGE)) of buffered extract of human skin cells (normal human epidermal keratinocytes (NHEK)) which had been exposed to mustard-type chemical compounds. The protease was identified as a band at approximately 50,000 to 80,000 daltons molecular weight. That protein was seen NHEK, in pig skin, and in skin of hairless mice that were exposed to mustard compounds. The mustard compounds used included chloroethyl ethyl sulfide (CEES), Bis-2-chloroethyl sulfide, also known as sulfur mustard, and 2-chloro-N-(2-chloroethyl)-N-methylethanamine hydrochloride, also known as nitrogen mustard (HN₂). The protein band from NHEK cultures had protease activity that was identical to characteristics of protease stimulated by mustard compounds in other systems.

The '108 patent goes on to define the identified protein band as a biomarker of exposure to vesicant compounds such as these. The marker protein was suggested to be utilizable either to raise protective antibodies to protect against the protease or used in a kit for identifying presence or absence of the marker in the study of tissues taken from individuals who may have been exposed to mustard poisoning. At present, pathological changes resulting from mustard poisoning are identified by histopathologic and electron microscopic means, and possibly by the method of the '108 patent. The marker identified according to the '108 patent appears to be a band identifiable on a polyacrylamide gel. Also, the increase in the intensity of staining of the band on a polyacrylamide gel correlates with an increase in activity of a protease that requires calcium ion for activity and which is inhibited by leupeptin, but not pepstatin at 1 mM concentrations.

Another aspect of the present invention regards the treatment of conditions in which the expression of ADAM10 is deleterious. U.S. Pat. No. 6,228,648 (the '648 patent) discloses a method for modulating expression of ADAM10 expression through contact of cells exposed to a vesicant with nucleic acid molecules encoding the anti-sense, and thereby inhibiting the expression of, ADAM10.

The '648 patent describes the ADAM (“A Disintegrin And Metalloproteinase”) family of mammalian zinc metalloproteinases as “a recently characterized group of membrane bound proteins which have been implicated in cell adhesion and extracellular matrix proteolysis. These proteins have also been referred to as MDCs (metalloproteinase/disintegrin/cysteine-rich), cellular disintegrins, disintegrin-metalloproteinases and metalloproteinase-disintegrins.”

According to the '648 patent, all ADAM family members display a common domain organization and are unique among cell surface proteins in possessing both a potential adhesion domain as well as a potential protease domain. It is stated that since “cell surface proteolysis plays an important role in development and pathology” and asserts that “so few cell-surface membrane-anchored proteases have been described,” thus leading to the conclusion that ADAMs play important roles in development and disease (Stone, et al., J. Protein Chem., 1999, 18, 447-65).

These Zn-activated metalloproteinases which also adhere to cells via “disintegrins”, referred to in the '648 as “ADAMs”, are reportedly expressed in a wide range of animal species, tissues and cell types and have been implicated in sperm-egg fusion, spermatogenesis, neutrophil infiltration, platelet aggregation, neurogenesis and cachexia. In addition, ADAMs have been shown to be involved in proteolysis of membrane anchored cytokines, growth factors and their receptors (Wolfsberg et al., J. Cell. Biol., 1995, 131, 275-8; Yamamoto et al., Immunol. Today, 1999, 20, 278-84).

Historically, ADAM10 (also known as MADM, HuAD10 and kuzbanian) was originally isolated from bovine brain and shown to act as a metalloproteinase involved in the degradation of myelin basic protein (Howard et al., Biochem. J., 1996, 317, 45-50). This same enzyme was also shown to act as a type IV collagenase in the bovine kidney where it was shown to cleave a human placental basement membrane collagen preparation (Millichip et al., Biochem. Biophys. Res. Commun., 1998, 245, 594-8). In human cells, ADAM10 was first identified in THP-1 membrane extracts. Here it was shown to cleave a peptide substrate of tumor necrosis factor (TNF) alpha (Rosendahl et al., J. Biol. Chem., 1997, 272, 24588-93). Subsequently, it has been shown to act as a convertase with the ability to cleave both a recombinant pro-TNF-alpha and a synthetic peptide to the mature TNF-alpha protein (Lunn et al., FEBS Lett., 1997, 400, 333-5).

In the skeletal system, ADAM10 is expressed in specific regions of articular cartilage and metaphyseal bone of the neonatal rat tibia. In the bone and cartilage, it is expressed as two different transcripts of 4.5 kb and 7.5 kb by periosteal cells, osteoblasts and osteocytes at areas of active bone formation (McKie et al., Biochem. Biophys. Res. Commun., 1997, 230, 335-9; Dallas et al., Bone, 1999, 25, 9-15). These two isoforms are believed to be generated by the mechanisms of alternative splicing and/or alternative polyadenylation. In primary human osteoblasts and osteoblast cell lines, ADAM10 is localized in the trans-Golgi network and on the plasma membrane (Dallas et al., Bone, 1999, 25, 9-15).

More recently it has been demonstrated that the levels of ADAM10 mRNA are elevated in osteoarthritis tissues. These levels appear to be related to the degree of cartilage damage and/or degradation that suggests a role for ADAM10 in cartilage matrix destruction associated with osteoarthritis (Chubinskaya et al., J. Histochem. Cytochem., 1998, 46, 723-9).

ADAM10 has also been identified as an autoantigen in a patient with pulmonary fibrosis associated with dermatomyositis (Fujita et al., Ann. Rheum. Dis., 1999, 58, 770-2). Pulmonary fibrosis is an inflammatory disease of the lung.

In studies of cell lines from hematologic malignancies such as leukemia, erythroleukemia, lymphoma, and myeloma, ADAM10 expression was shown to be significantly elevated (Wu et al., Biochem. Biophys. Res. Commun., 1997, 235, 437-42). Here again, two transcripts were observed by Northern blot analysis.

In light of all of these studies, the '648 patentees assert that “pharmacological modulation of ADAM10 activity and/or expression is therefore believed to be an appropriate point of therapeutic intervention in pathological conditions such as connective tissue disorders, inflammation and hematologic malignancies.”

The '648 patent asserts: “Currently, there are no known therapeutic agents which effectively inhibit the synthesis of ADAM10 and to date, strategies aimed at investigating ADAM10 function have involved the use of antibodies to localize ADAM10 (Millichip et al., Biochem. Biophys. Res. Commun., 1998, 245, 594-8). These antibodies were not shown to inhibit ADAM10 function and/or activity. Consequently, there remains a long felt need for agents capable of effectively inhibiting ADAM10 function.”

The solution proposed by the '648 patent is the use of antisense technology, which is asserted to be “emerging as an effective means for reducing the expression of specific gene products and may therefore prove to be uniquely useful in a number of therapeutic, diagnostic, and research applications for the modulation of ADAM10 expression.”

Thus, there is a need for compositions and methods that treat, reduce the adverse effects or, and otherwise aid in the healing of exposure to vesicating chemicals such as mustard compounds and similarly acting agents, and to modulate the adverse effects of ADAM10 expression in a number of disease conditions.

The present invention provides compositions and methods that treat, reduce the adverse effects or, and otherwise aid in the healing of exposure to vesicating chemicals such as mustard compounds (also referred to as mustard and mustard chemical) and similarly acting agents. The present invention also provides compositions and methods for modulating the adverse effects of ADAM10 expression, when needed, including modulation of the alternatively spliced form of ADAM10. These compositions and methods utilize a now known or hereafter identified matrix metalloproteinase inhibitory compound (MMPI), preferably in combination with protease inhibitor such as a serine protease inhibitor, optionally with other disclosed biologically active compounds, such as an anti-inflammatory agent (AIA), biologically inert compounds, and combinations thereof.

MMPIs are known in the art to include a number of compositions, and have been used to treat various conditions in which the activity of matrix metalloproteinases is sought to be reduced. For example, rheumatoid arthritis, keratoconus, alkali burns, and other conditions and diseases are mentioned as relevant situations in which an MMPI may be useful to treat such condition. Examples of patents that disclose MMPIs, their production and use are U.S. Pat. Nos. 5,114,953, 5,183,900, 5,773,438, and 5,892,112.

These patents, and all other patents, patent applications and publications discussed or cited herein are understood to be incorporated by reference to the same extent as if each individual publication or patent application was specifically and individually set forth in its entirety.

### SUMMARY OF THE INVENTION

A matrix metalloproteinase inhibitor, MMPI, in an appropriate vehicle, has been found to provide superior results in treating vesicant-exposed eyes in a rodent eye model. One embodiment of the present invention comprises an MMPI, in an appropriate vehicle, in combination with at least one traditionally-recognized anti-inflammatory compound, and optionally with the MMPI also exhibiting some type of anti-inflammatory effect.

### DETAILED DESCRIPTION OF THE PREFERRED EMBODIMENTS

This invention relates to the discovery that toxicity to mustard and other vesicants may be reduced, prevented or avoided by treatment, preferably before, but also upon and subsequent to exposure to vesicant compounds, with either: an MMPI alone; or an MMPI in combination with an anti-inflammatory compound, and/or having an anti-inflammatory effect; according to a regimen disclosed herein. Another aspect of the present invention is the use of an MMPI, optionally comprising an anti-inflammatory agent, to treat injuries caused by acids and bases. These compositions of the present invention represent an improvement in the treatment of such injuries.

The matrix metalloproteinase inhibitors (MMPIs) for use according to this invention include, but are not limited to, those MMPI compounds disclosed and claimed in U.S. Pat. Nos. 5,114,953, 5,183,900, 5,773,438, and 5,892,112. U.S. Pat. No. 5,183,900 provides a definition of a mammalian matrix metalloproteinase, which is specifically incorporated by reference to indicate the nature of a typical matrix metalloproteinase.

In addition to the MMPI, it is desirable that an anti-inflammatory agent (AIA) is included in the composition. As used in this disclosure, an anti-inflammatory agent is taken to include members of the three general classes of traditionally-recognized AIAs: aspirin; steroidal anti-inflammatory agents, and non-steroidal anti-inflammatory agents. Agents exhibiting anti-inflammatory activity useful according to this invention include, but are not limited to, hydrocortisone, triamcinolone, indomethacin, dexamethasone, and like compounds.

In addition, agents that act on specific pathways to work against an inflammatory response, but which are not included in these three traditional classifications of AIAs, are also considered to fall within the definition of an AIA for the purposes of this disclosure. For instance, it is known that Ilomastat, a recognized MMPI, also exhibits anti-inflammatory activity due to it ability to inhibit the TNF-alpha converting enzyme, which prevents release of soluble TNF-alpha. Ilomastat is the ICAN name for the chemical identified as CAS-142880-36-2, whose chemical formula is C₁₈H₂₈N₄O₄, and having a molecular weight of 388.46.

A preferred embodiment of the present invention is the use of a single molecule has both anti-inflammatory activity and also acts as an MMPI. Thus, the use of Ilomastat, which possesses both MMPI and AI properties, is preferred in one embodiment of the present invention.

For application to the eye, it is desirable to include the MMPI and AIA (and/or the multi-function compounds above) in an ocularly acceptable carrier, such as are known in the art, with an amount of the MMPI and AIA effective to prevent, reduce, avoid or eliminate the adverse effects of vesicant exposure to the eye. For application to the skin, or about an injury or abrasion in the skin, it is desirable to include the MMPI and AIA in a dermally acceptable carrier, such as are known in the art, with an amount of the MMPI and AIA effective to prevent, reduce, avoid or eliminate the adverse effects of vesicant exposure to the skin.

Known carriers for the eye are lubricious, non-toxic and preserve the pH, osmotic and chemical balance of the eye. Known carriers for the skin are various creams and solutions, many of which are also adapted for facilitating transdermal penetration of the biologically effective agent included in the applied composition.

For each component, an effective amount is that amount correlative with the amount of matrix metalloproteinase and inflammation anticipated to be induced by exposure to the vesicant. Concentrations of about 10 ng/mL to about 100 mg/mL of the MMPI are preferred. The AIA, if present, is desirably present at a concentration that controls the inflammatory component of exposure to a given vesicant, and may be present in the composition of this invention at a concentration of between 1000:1 to about 1:1000 in comparison to the concentration of vesicant to which the eye or skin is exposed. Preferably, the AIA is present in the composition at a concentration of about 10 ng/mL to about 100 mg/mL, depending on such routinely defined parameters as solubility and dose response requirements upon exposure to known vesicants.

A rabbit eye blister model is used to study and characterize tissue destruction induced by some common mustards, including 1-chloroethyl ethyl sulphide (CEES) and 2-chloro-N-(2 chloroethyl)-N-methylethanamine hydrochloride (HN₂), or other known vesicants, such as certain acids and bases, and the like. Rabbit eyes provide an appropriate model that makes it possible to perform the experimental manipulations necessary to study the effect of vesicants and the efficacy of the MMPI composition according to this invention.

In light of the general disclosure provided herein above, with respect to the manner of practicing this invention, and the manner of making the composition according to this invention, those skilled in the art will appreciate that this invention enables the practice of the invention as defined in the attached claims. However, the following experimental details are provided to ensure a complete written description of this invention, including of the best mode thereof. However, it will be appreciated that the scope of this invention should not be construed in terms of the specific examples provided. Rather, the scope of this invention is to be apprehended with reference to the claims appended hereto, in light of the complete description of this invention constituted by this entire invention disclosure.

### Example 1

### Rabbit Eye Model for Demonstrating Effect of MMPI

Rabbit eyes are treated with (a) vehicle alone, and (b) Ilomastat.

### Example 2

Effect of the composition of this invention at different concentrations of vesicant, such as mustard chemical.

Rabbit eyes are exposed to different concentrations of mustards, with the dose of the composition according to this invention varied, so as to achieve a dose-response curve, for each specific vesicant tested, as well as an optimal concentration of the composition of this invention for a given vesicant concentration, by monitoring the reduction in tissue damage achieved.

### Example 3

### Combined Effect of Composition of this Invention Including an Anti-Inflammatory Agent

The experiments described in Examples 1 and 2 are conducted in the presence of an anti-inflammatory agent. The degree of total inflammation at different concentrations of vesicant at optimal concentrations of the composition of this invention for the given vesicant is monitored. It is found that addition of anti-inflammatory agents to MMPI and AIA (whether provided as separate molecules or a single molecule) significantly reduces the total degree of inflammation as compared to vesicant alone, vehicle alone, or anti-inflammatory agent alone.

Having generally described this invention, including the best mode thereof, those skilled in the art will appreciate that the present invention contemplates the embodiments of this invention as defined in the following claims, and equivalents thereof. However, those skilled in the art will appreciate that the scope of this invention should be measured by the claims appended hereto, and not merely by the specific embodiments exemplified herein. Those skilled in the art will also appreciate that more sophisticated technological advances will likely appear subsequent to the filing of this document with the Patent Office. To the extent that these later developed improvements embody the operative principles at the heart of the present disclosure, those improvements are likewise considered to come within the ambit of the following claims.

What is claimed is:

1. A composition for reducing morbidity induced by contact of tissue with a vesicant, wherein said composition comprises at least one matrix metalloproteinase inhibitor (MMPI).

2. The composition according to claim 1 further comprising an anti-inflammatory agent, AIA.

3. The composition according to claim 2 wherein said AIA is hydrocortisone, triamcinolone, indomethacin, dexamethasone, or a combination thereof.

4. The composition according to claim 1 which is an ocular composition or a composition adapted for administration to the skin.

5. The composition according to claim 2 which is an ocular composition or a composition adapted for administration to the skin.

6. The composition according to claim 1 comprising a molecule exhibiting both MMPI activity and anti-inflammatory activity.

7. The composition according to claim 6 wherein said molecule is Ilomastat.

8. The composition according to claim 1 wherein said composition comprises concentrations of about 10 ng/mL to about 100 mg/mL of the MMPI.

9. The composition according to claim 2 wherein said AIA is present at a concentration that controls the inflammatory component of exposure to a given vesicant, and is present in the composition of this invention at a concentration of between 1000:1 to about 1:1000 in comparison to the concentration of vesicant to which the eye or skin is exposed.

10. The composition according to claim 2 wherein said AIA is present in the composition at a concentration of about 10 ng/mL to about 100 mg/mL.

11. A method for minimizing the adverse effects on tissue of exposure to a vesicant, comprising contacting said tissue, prior to, concurrent with, or subsequent to exposure to said vesicant with a matrix metalloproteinase inhibitor, MMPI.

12. The method according to claim 11 wherein the MMPI is Ilomastat.

13. The method according to claim 11 wherein said method further comprises contacting said tissue with an anti-inflammatory agent (AIA) either prior to, concurrent with or subsequent to contacting said tissue with said MMPI.

14. The method according to claim 13 wherein said contacting comprises administering said MMPI and said AIA in an ocular vehicle to the eye or administering said MMPI and said AIA in a dermal preparation.

15. The method according to claim 14 wherein said vesicant is 1-chloroethyl ethyl sulphide (CEES), 2-chloro-N-(2 chloroethyl)-N-methylethanamine hydrochloride (HN₂), or a combination thereof.

16. A method to treat exposure to vesication-causing chemical agents in an animal, said method comprising administering to an animal suffering from said disease an effective amount, and for an effective time a synthetic mammalian matrix metalloproteinase inhibitor.

17. The method according to claim 16, wherein the method further comprises administering to the animal an effective amount and for an effective time an anti-inflammatory agent.

18. The method according to claim 16, wherein the synthetic mammalian matrix metalloproteinase inhibitor is Ilomastat.
